(19) United States
(12) Patent Application Publication
(10) Pub. No.: US 2026/0199191 A1
(43) Pub. Date: Jul. 16, 2026

(54) COATED SUBSTRATES AND DELAMINATION OF PATHOGENIC FORMATIONS THEREFROM

(71) Applicants: Arizona Board of Regents on behalf of Arizona State University, Scottsdale, AZ (US); Nanobionics LLC, Phoenix, AZ (US)

(72) Inventors: Hanna Bandarenka, Chandler, AZ (US); Bruno Azeredo, Scottsdale, AZ (US); Nayfs Samandari, Phoenix, AZ (US)

(21) Appl. No.: 19/135,130

(22) PCT Filed: Dec. 12, 2022
(86) PCT No.: PCT/US2022/052550

§ 371 Date: Jun. 3, 2025

(51) Int. Cl. A61K 6/20; A61K 6/17; A61K 8/19; A61Q 11/00

(52) U.S. Cl. CPC A61K 6/20; A61K 6/17; A61K 8/19; A61Q 11/00; A61K 2800/413; A61K 2800/621; A61K 2800/88

(57) ABSTRACT

Methods are provided for cleaning a substrate. An exemplary method includes irradiating a pathogenic formation on a surface of the substrate with radiation in a wavelength range of 350 nm to 800 nm, wherein the substrate includes metal particles on the surface of the substrate, and wherein a highest temperature of the metal particles during the irradiation is less than or equal to 45° C.; and delaminating the pathogenic formation from the substrate.

### TECHNICAL FIELD

This disclosure relates to coated substrates and delamination of pathogenic formations from the coated substrates.

### BACKGROUND

Dental surfaces are a harbor for many pathogenic microorganisms such as bacteria, viruses, fungi, dead cell structures and residual food particles. This leads to the formation of dental plaque (biofilm), which is easily generated within the oral environment in less than 2 hours after a professional oral cleaning. Biofilm becomes the cause of the premature failure of the restoration and the source of many local and systemic disease processes that affects the health of patients. Similar biofilm hazards occur on natural teeth leading to the failure of the tooth, which can require a dental restoration, a root canal cleaning, or a tooth replacement with an implant.

Once formed, the biofilm is attached via weak interatomic bonds between atoms of molecules in the synthetic or natural dental surfaces and of molecules present in the adherent pathogenic formation. Although the growing pathogenic formation is anchored to the dental object via weak bonds, an enormously strong adhesion develops and increases over time because of micro- and nanoscaled roughness of both the synthetic and natural surfaces. Over time, plaque can become mineralized because of acidic secretions from the plaque itself that demineralize adjacent tooth surfaces and, if not treated properly, can be converted to tartar, which is connected with the dental surfaces even more strongly. The plaque matrix is mostly constituted of polysaccharides, proteins, nucleic acids and lipids that form a polymeric network responsible for the stability of plaque structure and its increased adhesion to the host surfaces. As noted above, increasing adherence and stagnation become a danger to the oral and general health of the patient. Over the time, it becomes increasingly difficult to remove the plaque without the use of aggressive chemical treatments or professional dental treatments. Even with regular access to professional dental cleanings, the intermittent periods for patients between such treatments are detrimental to their overall health and aesthetics. This is because the pathogens in the oral environment invade the gingival tissues surrounding dental implants or restorations and even cause infections into the nerve structures of the teeth. The unchecked infections lead to abscessed teeth roots, in which canals are filled and covered with infected necrotic tissues, making them an ideal source for biofilm growth.

Therefore, a need exists to develop new techniques and surface coatings to both prevent and reduce the growth of biofilm.

### BRIEF SUMMARY

Provided herein is a method of cleaning a substrate, the method including: irradiating a pathogenic formation on a surface of the substrate with radiation in a wavelength range of 350 nm to 800 nm, wherein the substrate comprises metal particles on the surface of the substrate, and wherein a highest temperature of the metal particles during the irradiation is less than or equal to 45° C.; and delaminating the pathogenic formation from the substrate.

In some embodiments, an average diameter of the metal particles is in a range between 200 nm and 2000 nm.

In some embodiments, the metal particles comprise silver.

In some embodiments, a shortest distance between exterior surfaces of adjacent metal particles is at least 10 nm.

In some embodiments, a density of the metal particles is in a range of 0.1 to 30 metal particles per 1 μm² of the substrate surface. In some embodiments, a density of the metal particles is in a range of 1 to 10 metal particles per 1 μm² of the substrate surface.

In some embodiments, a density of the metal particles is selected based on an average diameter of the metal particles. In some embodiments, an optimal density of the metal particles varies inversely with the average diameter of the metal particles.

In some embodiments, a density of the metal particles is in a range of 1 to 10 metal particles per 1 μm² of the substrate surface when the metal particles have an average diameter in a range of 200 nm to 1000 nm. In some embodiments, a density of the metal particles is in a range of 1 to 10 metal particles per 1 μm² of the substrate surface when the metal particles have an average diameter in a range of 200 nm to 600 nm. In some embodiments, a density of the metal particles is in a range of 3 to 5 particles per 1 μm² of the substrate surface when the metal particles have an average diameter in a range of 400 nm to 500 nm. In some embodiments, a density of the metal particles is in a range of 5 to 10 particles per 1 μm² of the substrate surface when the metal particles have an average diameter in a range of 200 nm to 300 nm.

In some embodiments, the irradiating occurs for a length of time in in a range of about 0.1 seconds to about 30 minutes.

In some embodiments, the irradiating increases a temperature of the metal particles by 25° C. or less, 20° C. or less, 15° C. or less, 10° C. or 5° C. or less.

In some embodiments, during the irradiating, the metal particles do not undergo surface plasmon resonance.

In some embodiments, the method further comprises disposing the metal particles on the substrate. In some embodiments, disposing the metal particles on the substrate comprises spraying the metal particles on the substrate. In some embodiments, disposing the metal particles on the substrate comprises immersion deposition or chemical deposition.

In some embodiments, the substrate is a tooth or a dental crown. In some embodiments, the tooth is a natural tooth or a dental implant.

In some embodiments, delaminating comprises rinsing the substrate to remove the pathogenic formation from the substrate.

In some embodiments, the pathogenic formation comprises a biofilm.

Also provided herein is a coated substrate including: a substrate, and metal particles on the surface of the substrate. The metal particles have an average diameter in a range of 200 nm and 2000 nm, wherein a shortest distance between exterior surfaces of adjacent metal particles is at least 10 nm, and a density of the metal particles is in a range of 0.1 to 30 metal particles per 1 μm² of the substrate surface.

Also provided herein is a kit including: a pretreatment composition including a reducing agent; and a coating agent including metal ions

In some embodiments, the coating agent comprises silver nitrate.

In some embodiments, the reducing agent comprises potassium sodium tartrate.

In some embodiments, the kit further comprises a light source configured to be inserted in the mouth of a user.

The methods disclosed herein provide removal of pathogenic formations formed on surfaces without requiring the use of high temperatures. The methods disclosed herein are also advantageous because the delamination of pathogenic formations can be achieved without disruption of the structure of the pathogenic formation. Rather, the intact pathogenic formation may be removed in the delamination process, which can prevent the release of undesirable microorganisms that may lead to re-infection of the oral cavity.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF THE DRAWINGS

FIG. 1A is a schematic representation of a pathogenic formation on a natural or synthetic dental substrate and coupled to the substrate via weak secondary bonds. FIG. 1B depicts the dental substrate of FIG. 1A with metal particles on the surface of the substrate.

FIG. 2 is a flowchart of steps of a method for delamination of pathogenic formations from dental substrates including methods that apply to the manufacturing and creation of the coated dental surfaces and dental clinics together with steps performed by the patient who has been treated pursuant to the method disclosed herein or has received a dental prosthetic.

FIG. 3 shows comparison of dental objects (crowns) subjected to all the steps of the method shown in FIG. 2 and those for which steps 1-2 or step 4 were missed. The delamination of the pathogenic formation (plaque) from the crown subjected to the proposed method is observed in contrast to crowns that did not contain silver (Ag) particles or were not excited with light.

FIGS. 4A and 4B show scanning electron microscopy (SEM) top images of a dental object (crown) before (FIG. 4A) and after (FIG. 4B) growth of Ag particles. The diameter of Ag particles varies from 230 to 450 nm. The number of Ag particles per 1 μm² varies from 4 to 8 particles.

FIG. 5A shows a scanning electron microscopy (SEM) top image of the dental surface (ZrO₂ crown) after growth of Ag particles. FIGS. 5B and 5C show corresponding results of elemental analysis of the dental surface (ZrO₂ crown) after growth of Ag particles made by energy dispersive X-ray spectroscopy (EDS) scanning. The crown was partially covered with SiO₂ dental glaze (right area in FIGS. 5A and 5B). FIG. 5C shows distribution of Ag (light dots) on the dental surface and demonstrate that Ag particles uniformly coated entire dental surface independently on its composition (ZrO₂ or SiO₂).

FIGS. 6A and 6B show scanning electron microscopy (SEM) top images of a dental object (tooth, molar) before (FIG. 6A) and after (FIG. 6B) growth of Ag particles by chemical deposition of Ag from solution of AgNO₃, glucose, NaOH, NH₄OH, and H₂O. The size of Ag particles varies from 200 to 550 nm. In rare spots, it reaches 1-2 μm due to particle coalescence. The number of Ag particles per 1 μm² varies from 1 to 10 particles.

FIG. 7A shows polymer film, polymer film with Ag particles, ZrO₂ crown and ZrO₂ crown with Ag particles before and after excitation with LED (visible wavelength) for 30 minutes. The Ag particles of the polymer film shown on the left demonstrate SERS-activity due to surface plasmon resonance (SPR), and are referred to herein as “SPR particles.” The SPR particles were formed by deposition of Ag on Si-based substrate and then transferred to polymer film to avoid heat dissipation via Si. The Ag particles of the ZrO₂ crown shown on the right are those presented in FIG. 4. The particles of the polymer film shown on the left are heated up by about 31° C. The Ag particles of the ZrO₂ crown shown on the right have much lower temperature change, and are referred to herein as “non-SPR particles.” Differences between SPR particles and non-SPR particles include properties of the particles (e.g., particle size) as well as properties of the particle distribution (e.g., particle density).

FIG. 7B shows polymer film, polymer film with Ag particles, ZrO₂ crown and ZrO₂ crown with Ag particles before and after excitation with laser (445 nm) for different periods of time (maximal—20 minutes). The SPR particles of the polymer film shown on the left are heated up to 54° C. from an initial temperature of 19.3° C., showing an increase in temperature by 34.7° C. The non-SPR Ag particles of the ZrO₂ crown shown on the right have much lower temperature change.

FIG. 8 shows the surface potential of a ZrO₂ crown and a ZrO₂ crown coated with silver particles versus time. Zero and four minute points on the time axis correspond to turning on and turning off a light emitting diode of a dental device, respectively.

FIG. 9 shows Raman (a) and SERS spectra (b and c) of Ellman's reagent (5,5′-dithio-bis-[2-nitrobenzoic acid]) at concentration (a) 10⁻¹ M on glass, (b) 10⁻⁵ M on Ag SPR particles and (c) Ag non-SPR particles on ZrO₂ dental object (crown).

FIG. 10 depicts an example of a kit including a pretreatment composition and a coating agent.

FIG. 11A depicts an example of a kit including a pretreatment composition, a coating agent, and optional components such as an LED light source configured to be inserted in the mouth of a user. FIG. 11B depicts an example of a kit including a pretreatment composition, a coating agent, and optional components such as a laser light source configured to be inserted in the mouth of a user.

### DETAILED DESCRIPTION

As used herein, a “pathogenic formation” refers to bacteria, viruses, fungi, dental plaque (biofilm), tartar, dead cell structures, necrotic tissue (e.g. dead nerve and abscess tissue in a root canal), residual food particles, or a combination thereof. In some embodiments, the pathogenic formation includes a biofilm.

As used herein, “dental surfaces” refer to the surface portion of natural teeth, dental restorations, synthetic implants, reconstructions, periodontal and orthodontic appliances, crowns, exposed portions of dental implants used for permanent replacement of natural teeth, the facial, lingual, occlusal, and interproximal portion of any natural or synthetic implant that would benefit from delamination of pathogenic formation. Portions of natural teeth and roots following root canals and other therapeutic procedures that expose any portion of natural or synthetic surfaces to susceptibility of pathogenic formations.

As used herein, “secondary bonds” refer to bonds that form from intermolecular forces of attraction, including but not limited to electrostatic forces, steric forces, and van der Waals bonds, including fluctuating induced dipole bonds, polar molecule-induced dipole bonds, and permanent dipole bonds that are susceptible of being distracted by the application of a directed light source.

Provided herein is a method of cleaning a substrate. The method includes irradiating a pathogenic formation on a surface of the substrate, wherein the substrate comprises metal particles on the surface of the substrate; and delaminating the pathogenic formation from the substrate.

Also provided herein is a method of delaminating pathogenic formations including but not limited to plaque, biofilms, tartar, necrotic tissue, or combinations thereof, from a substrate. The method can include coating a substrate with metal particles, and subsequently irradiating the substrate, the pathogenic formation, or the particles with energy from a incoherent or coherent light source.

In some embodiments, the substrate includes components of functional nanomaterials (e.g., active areas of optical sensors), medical implants, medical tools (e.g., surgical instruments), dishware (plates, cups, cutlery, pots dishes, etc.), or a dental substrate.

In some embodiments, the substrate is a dental substrate including but not limited to natural teeth, walls of teeth root canals, crowns, dentures, and dental appliances. In some embodiments, the substrate is a tooth or a dental crown. In some embodiments, the tooth is a natural tooth or a dental implant. The dental substrates to which the metal particles are applied include any of synthetic prosthetic devices manufactured from metals, metal oxides (e.g., titanium, zirconium, aluminum oxides), dental ceramics (e.g., silica), natural surfaces (e.g., hydroxyapatite), and combinations of natural dental surfaces that have been completely or partially restored to incorporate dental medicals, composites, and other commonly used dental materials. The materials can be doped with other elements like rare earth metals or their compounds. The intended dental surface can be structural portions of a prosthetic, such as a portion of a dental implant, orthodontic device, or other temporary or permanent prosthetic, or can be purely aesthetic structures such as veneers, crowns, or prosthetic crown portions of dental implants that resemble natural human teeth.

The material of the dental substrate can be metals, pure metal oxides, doped metal oxides, any amorphous substance, dental plastics assembled from organic and/or inorganic molecules including natural composites of minerals and organic molecules in teeth enamel, and necrotic and living tissues. In some embodiments, the substrate is a silicon-based substrate. In some embodiments, the substrate is a highly doped monocrystalline n-Si wafer. In some embodiments, the substrate is a zirconia-based substrate. In some embodiments, the substrate is a yttrium doped zirconia dental crown.

In some embodiments, the substrate surface may be mechanically or electrochemically nanoroughened before the metal particles are applied.

In some embodiments, the metal particles comprise silver, gold, copper, platinum, palladium, nickel, chromium, cobalt, titanium, stainless steel, aluminum, zinc, and/or other metals applied in dentistry. In some embodiments, the metal particles comprise silver. In some embodiments, the metal particles are silver particles. In some embodiments, the metal particles comprise metal nanoparticles.

In some embodiments, the method further comprises disposing the metal particles on the substrate. The metal particles may be applied to the surface of the substrate by immersion, chemical, or magnetron sputtering techniques. In some embodiments, disposing the metal particles on the substrate comprises spraying the metal particles on the substrate. In some embodiments, disposing the metal particles on the substrate comprises immersion deposition or chemical deposition. In some embodiments, the metal particles are applied by the deposition of silver, gold, copper, platinum, palladium, nickel, chromium, cobalt, titanium, stainless steel, aluminum, zinc, and/or other metals applied in dentistry.

In some embodiments, an average diameter of the metal particles is in a range between 200 nm and 2000 nm, such as between 200 nm and 1000 nm, between 200 nm and 500 nm, between 150 nm and 2000 nm, or between 200 nm and 750 nm. In some embodiments, an average diameter of the metal particles is in a range between 200 nm and 2000 nm. In some embodiments, an average diameter of the metal particles is in a range between 200 nm and 1000 nm. In some embodiments, an average diameter of the metal particles is at least 200 nm. In some embodiments, an average diameter of the metal particles is below 2000 nm.

In some embodiments, a shortest distance between exterior surfaces of adjacent metal particles is at least 10 nm. In some embodiments, a density of the metal particles is in a range of 0.1 to 30 metal particles per 1 μm² of the substrate surface. For example, a range of from 0.2 to 25 metal particles per 1 μm², from 0.24 to 22 metal particles per 1 μm², from 1 to 20 metal particles per 1 μm², from 1 to 15 metal particles per 1 μm², from 1 to 10 metal particles per 1 μm², from 2 to 10 metal particles per 1 μm², from 4 to 8 metal particles per 1 μm², from 3 to 5 metal particles per 1 μm², or from 5 to 10 metal particles per 1 μm². In some embodiments, the density of the metal particles is in a range of 1 to 10 metal particles per 1 μm² of the substrate surface.

In some embodiments, a density of the metal particles is selected based on an average diameter of the metal particles. In some embodiments, an optimal density of the metal particles varies inversely with the average diameter of the metal particles. In some embodiments, a density of the metal particles is in a range of 1 to 10 metal particles per 1 μm² of the substrate surface when the metal particles have an average diameter in a range of 200 nm to 1000 nm. In some embodiments, a density of the metal particles is in a range of 1 to 10 metal particles per 1 μm² of the substrate surface when the metal particles have an average diameter in a range of 200 nm to 600 nm. In some embodiments, a density of the metal particles is in a range of 1 to 10 metal particles per 1 μm² of the substrate surface when the metal particles have an average diameter in a range of 400 nm to 500 nm. In some embodiments, a density of the metal particles is in a range of 1 to 10 metal particles per 1 μm² of the substrate surface when the metal particles have an average diameter in a range of 200 nm to 500 nm. In some embodiments, a density of the metal particles is in a range of 3 to 5 metal particles per 1 μm² of the substrate surface when the metal particles have an average diameter in a range of 200 nm to 1000 nm, 300 nm to 800 nm, 400 nm to 750 nm, 400 nm to 600 nm, or 400 nm to 500 nm. In some embodiments, a density of the metal particles is in a range of 3 to 5 metal particles per 1 μm² of the substrate surface when the metal particles have an average diameter in a range of 400 nm to 500 nm. For example, a density of 4 metal particles per 1 μm² of the substrate surface when the metal particles have an average diameter of about 450 nm. In some embodiments, a density of the metal particles is in a range of 5 to 10 particles per 1 μm² of the substrate surface when the metal particles have an average diameter in a range of 200 nm to 1000 nm, 200 nm to 600 nm, 200 nm to 400 nm, or 200 nm to 300 nm. In some embodiments, a density of the metal particles is in a range of 5 to 10 particles per 1 μm² of the substrate surface when the metal particles have an average diameter in a range of 200 nm to 300 nm. For example, a density of 8 metal particles per 1 μm² of the substrate surface when the metal particles have an average diameter of about 230 nm.

In some embodiments, a density of the metal particles over 10 nm is less than 10 in 1 μm² of the substrate surface, e.g., for 2 μm particles their number in 10 μm² of substrate surface can be 16. In some embodiments, the metal particles do not provide high frequency of free electron oscillations leading to intensive SPR and to extreme light trapping.

In certain embodiments, the metal particles are applied to the substrate by the application of liquid compositions containing silver ions to a target dental surface followed by treatment with a reducing agent. This results in reduction of silver ions to silver atoms on the surface of the substrate and in the formation of a coating composed of silver particles. In the case of synthetic dental surfaces, a pretreatment for their roughening can be performed to provide stronger adhesion of the silver coating to them, which allows for longer shelf life of the coating. If natural teeth are subjected to the deposition of silver particles, they can be preliminary cleaned to remove any organic contaminations from their surface. There is no need for an additional pretreatment of the natural dental surface for improvement of the adhesion of silver particles since the natural teeth have rough surface. Exemplary pretreatment and cleaning procedures are described below. These procedures can be immersion of the synthetic dental surfaces into piranha solution or solutions containing fluoric ions for a short period (maximally a few minutes) and washing the natural teeth with diluted hydrochloric solutions. Application techniques include quick gentle brushing the natural dental surfaces with solutions providing reduction of silver ions to silver atoms, immersion of synthetic dental surfaces into those solutions, or filling root canals with them.

The disclosed method of chemical deposition of a coating made of silver particles from solutions containing ions of silver is typically easier, quicker and cheaper than other methods of chemical deposition. In certain embodiments, colloidal dispersions of silver particles can be used as the dispersion phase with a separate composition in the continuous phase to maintain the silver particles in substantially uniform dispersion prior to application to the dental surface. The continuous phase may be comprised of a dental glaze or other constituent solute that is applied to the dental surface and may become incorporated therein. As a result, the components of the continuous phase may be incorporated into the layer of the silver composition applied directly to the dental surface, or may be a coating applied in a superior orientation to the coated dental surface to act as a barrier to a layer containing the silver particles.

In some embodiments, the method includes applying a reducing agent to the substrate and then applying a solution with metal ions for a few minutes. In this way, metallic particles grow forming an uneven layer, which can preserve the original color of the restoration or teeth.

In some embodiments, the disposing the metal particles on the substrate is performed by immersion deposition of silver onto the substrate. The substrate is immersed in a liquid solution comprising silver ions. In some embodiments, the liquid solution comprises silver nitrate. In some embodiments, the liquid solution comprises water, ethyl alcohol, and hydrofluoric acid. In some embodiments, the solution is free of hydrofluoric acid. In some embodiments, the liquid solution comprises ammonia solution, water, sodium hydroxide, and potassium sodium tartrate. In some embodiments, the immersion is performed for about 2 hours. After deposition, the substrate can be removed from the solution, rinsed with deionized water, and dried with airflow.

In some embodiments, the substrate is coated with a metal particle film by magnetron sputtering and is then annealed. In some embodiments, the film has a thickness of from about 5 nm to about 100 nm, such as from about 10 nm to about 75 nm, such as form about 20 nm to about 50 nm. In some embodiments, the metal particle film has a thickness of about 20 nm. In some embodiments, the metal particle film has a thickness of about 50 nm. In some embodiments, the annealing is performed in an argon atmosphere at a temperature of about 150° C. to about 700° C. for about 5 minutes to provide silver film dewetting in particles. In some embodiments, after sputtering, the substrate is immersed in diluted nitric acid for about 0.5 h. In some embodiments, after sputtering, the substrate is rinsed with deionized water and dried with airflow.

After pathogenic formations are established on the surface of the substrate, the pathogenic formations, such as biofilm, can be removed by irradiating the pathogenic formation on the surface of the substrate. A pathogenic formation on a surface of the substrate is understood to be coupled to the substrate (e.g., in direct contact with the surface of the substrate or spatially separated from the surface of the substrate by an intervening layer (or layers) or a void). In one example, the pathogenic formation is formed over the metal particles that are disposed on the surface of the substrate. In some embodiments, the method includes irradiating a pathogenic formation with radiation in a wavelength range of from 350 nm to 800 nm. In some embodiments, irradiating a pathogenic formation is performed with radiation in a wavelength range of from 400 nm to 700 nm, 300 nm to 700 nm, 350 nm to 750 nm, 400 nm to 700 nm, 350 nm to 700 nm, 400 nm to 750 nm, 425 nm to 750 nm, 350 nm to 550 nm, 400 nm to 500 nm, 25 nm to 475 nm, 430 nm to 460 nm, or 445 nm to 450 nm.

The irradiation can occur for a length of time in a range of about 0.1 seconds to about 30 minutes. In some embodiments, irradiation occurs for a length of time in a range of about 1 second to about 30 minutes, about 5 seconds to about 30 minutes, about 1 second to about 15 minutes, about 1 second to about 10 minutes, about 0.5 seconds to about 5 minutes, or about 0.1 seconds to about 1 minute. In some embodiments, irradiation occurs for about 30 seconds. In some embodiments, irradiating includes exposure to a directed, intra oral light source. In some embodiments, irradiating is performed daily. In some embodiments, irradiating is performed twice daily. In some embodiments, irradiating is performed weekly.

The light source can be any commercially available light source, including a sealed or sterilizable unit commonly used in dentistry and adapted for consumer use, e.g. quartz-tungsten-halogen (QTH), light-emitting diode (LED), plasma arc curing (PAC), and Argon laser units. QTH light-curing units are the most widely used and are made of a quartz bulb containing a tungsten filament in a halogen environment. In some embodiments, the light source is existing LED versions with 400-500 nm incoherent light source with a power density about 1 mW/cm² or less, e.g. those for teeth whitening, or newly designed and engineered LED, e.g. light emitting diodes embedded in device to irradiate internal and external surfaces of teeth in oral cavity, tiny flexible optical fiber to penetrate in root canals. In some embodiments, the light source has a wavelength of from 400 nm to 500 nm, from 425 nm to 475 nm, from 430 nm to 460 nm, or from 445 nm to 450 nm. The power of the light on the surface of dental object can be very low, e.g. 0.1-1 mW/cm². Any other incoherent or coherent light source can be used if tuned to provide low power on the surface of dental object, e.g. use of optical filtering materials (polymers, glasses, etc.), defocusing, distancing up to 20 cm from dental object. The light source wavelength can be near ultraviolet, visible, or near infrared. Such a treatment will provide removal both aged and freshly formed plaque, tartar, and other pathogenic formations. In some embodiments, the light source is a blue dental LED device, a blue laser, or an LED projector having a wavelength in the visible range.

A feature of the method disclosed herein is that the irradiation used for the delamination can be non-intensive, i.e., there is no necessity to use powerful laser or light projector. This disclosure provides removal of multiple pathogenic formations formed on surfaces without the use of high temperatures. In the method described herein, the light source can use a relatively low power, can be incoherent, and is thus readily available and less expensive than the use of a high-power source such as a laser.

In some embodiments, irradiating the pathogenic formation increases a temperature of the metal particles by 25° C. or less, such as 20° C. or less, 15° C. or less, 10° C. or less, 5° C. or less, 3° C. or less, 2° C. or less, or 1° C. or less. In some embodiments, irradiating the pathogenic formation increases a temperature of the metal particles by 25° C. or less. In other words, the difference between an initial temperature of the metal particles and a highest temperature of the metal particles during the irradiation is 25° C. or less. In some embodiments, irradiating the pathogenic formation increases a temperature of the metal particles by 20° C. or less. In some embodiments, irradiating the pathogenic formation increases a temperature of the metal particles by 10° C. or less. In some embodiments, irradiating the pathogenic formation increases the temperature of the metal particles by 5° C. or less. In some embodiments, during the irradiating, the metal particles do not undergo SPR. In some embodiments, the intensity of the SPR bands for the particles is too low to cause a significant increase in a temperature of the metal particles even under resonance conditions.

In some embodiments, a highest temperature of the metal particles during the irradiation is less than or equal to 45° C., such as less than or equal to 40° C., less than or equal to 39° C., less than or equal to 38° C., less than or equal to 35° C., less than or equal to 30° C., or less than or equal to 25° C. In some embodiments, a highest temperature of the metal particles during the irradiation is less than or equal to 40° C.

Based at least on the properties and disposition of the metal particles as described herein, the application of the directed light source avoids heating that can damage living tissues, and thus differs from sterilization mechanisms for microorganisms wherein application of an excitation light frequency coupled with SPR of particles results in a photothermal effect that generates heat on the substrate surface.

Without wishing to be bound by theory, the irradiating step of the method described herein relates to creating the conditions to break weak secondary bonds between pathogenic formations and dental surfaces coated with particles. A proposed mechanism includes a charge polarization change induced by a low power light source. The irradiating is not accompanied by a significant increase in heat and thereby avoids causing damage to living tissues upon application of the directed light source, and thus differs in terms of application energy, the mechanism of the transfer of light energy to the dental surface, and in the absolute and relative heat created, from sterilization mechanisms for microorganisms where SPR particles are created by coupling SPR and excitation light frequencies to generate heat. The limited heating of the coated substrates disclosed herein upon light excitation is shown by measurements carried out with infrared camera.

In some embodiments, delaminating includes rinsing the substrate to remove the pathogenic formation from the substrate. In some embodiments, irradiating is followed by an oral rinse and subsequent removal of the pathogenic formation from a patient's mouth.

Without wishing to be bound by theory, delamination of pathogenic formations from the substrate can be achieved if secondary bonds between them will be broken in micro- and nano-scaled areas of the dental object surface. Without wishing to be bound by theory, the method described herein also enables the removal of biofilms from the under-surface of the colony versus from outer-surface of the colony. Most therapies in the oral armamentarium tackle the disease process via breaking through the top surface of biofilm and few have gone from the surface layer. Breaking the bond between the lower surface of the colony and the dental object surface is an advantage because it provides delamination of biofilm without the need to apply destruction to the entire volume of the biofilm, penetrating from its top to bottom.

The delamination step may be performed by the patient or consumer followed by removal of the pathogenic formations by mouth rinsing with an aqueous solution or solution that may include other agents to facilitate the delamination process or provide more effective removal of the pathogenic formations from the oral cavity of the patient. The delamination process may also be performed by a dental professional, but more typically the delamination process will be a part of a routine (e.g., daily, semi-daily, or weekly dental regimen) to reduce the gradual accumulation of pathogenic formations in the oral cavity of a patient.

The delamination step includes a non-traumatic removal of pathogenic formations from dental surfaces fixed on dental objects using techniques disclosed herein that are available for application by both patients and dental clinics. In such cases, the synthetic or natural dental surfaces can be applied to a fixture placed in the patient's mouth with instructions to perform a method of applying a light source to the metal particles in the coating for the delamination of pathogenic formations in a manner that cannot be achieved via conventional brushing, scraping, or ultrasound techniques.

Also provided herein is a method that applies a coating of silver particles on dental surfaces with high adhesion characteristics and that are manufactured in preparation for placement in the oral cavity. The coating can be applied by a dental professional with instructions to a patient for future application of a light source that delaminates the pathogenic formations.

Also disclosed herein is a method including providing a patient with a natural or synthetic coated dental surface and the capability to selectively activate a mechanism based on a directed light source. In some embodiments, the method breaks weak secondary bonds between pathogenic formations and dental surfaces coated with silver particles.

Also provided herein are alterations to conventional dental lab and dental office procedures to provide patients with synthetic and natural constructs containing dental surfaces such that can be delaminated by providing patients who have received prosthetic implants or treatment of natural dental surfaces with the metal particles of the present method with instructions and a light source to permit periodic and routine delamination of pathogenic formations from synthetic or natural dental surfaces previously provided to the patient.

In certain embodiments, synthetic prosthetics can be coated by a dentist through successive steps of quick immersion of the synthetic prosthetic or restoration in two or more different solutions containing a reducing agent and a solution containing silver ions, either in a colloidal solution followed by creating the coated dental surfaces by selectively applying the solution of silver particles to the natural or synthetic dental surfaces including incorporating and a dental glaze to coat existing dental crowns and bridges, as well as newly placed prosthetics and completely natural dental surfaces.

In one embodiment, silver particles can be grown in root canals and tubules for delamination of necrotic tissues. The directed light source can be provided by an incoherent or coherent light source, which power and/or wavelength are selected to both include excitation of the silver particles and to avoid heating of the metallic particles, while still causing interruption of weak secondary bonds between dental objects and pathogenic formations that further can be delaminated and removed during rinsing the oral cavity or root canal with an aqueous solution.

The methods disclosed herein are advantageous over previous methods that utilize thermal excitation of particles for destroying the biofilms via breaking strong chemical bonds in molecules composing biofilm matrix and microorganisms, because previous methods require heating up to tens or hundreds of degrees. Such a heating is dangerous for mechanical properties of dental objects and surrounding tissues. An additional advantage of the present disclosure is that delamination of pathogenic formation can be achieved without disruption of the structure of the formation, which does not necessarily lead to physical disruption of the biofilm that can cause releasing microorganisms undesirable re-infection of oral cavity. The intact delaminated biofilm, once separated, is expectorated after irradiation and rinsing of the mouth with water.

It is an advantage of embodiments of the methods disclosed herein proposed for growth of particles are cheap, fast, and compatible with current methods of dentistry and can be easily integrated into routine dental processes without the need for functionalization of surfaces of particles and dental objects.

Unlike surface treatments used in thermal applications, the present disclosure does not require that a patient or dentist use a powerful or specialized light source having a wavelength that coincides with SPR band of particles to create a threshold change in heat to reach a temperature great enough to cause for denaturation of proteins in microorganisms or disruption of strong chemical bonds in polymeric matrix of biofilm. A relatively low-level light source applied to the coated dental surface is enough to break the week, secondary bonds formed between the coated surface and the pathogenic formation thereby causing delamination and facilitating removal.

In some embodiments, because of the relatively low energy of the light source, no thermal, mechanical and other damage to dental objects occurs and there is no resultant change in the structure or function of the natural or synthetic surface and no operative changes in their physical or chemical properties.

The present disclosure also avoids the need for antibiotics or other chemical agents that aggressively kill microorganisms as well as for insertion of particles in pathogenic formations, which is complicated for daily dental care.

Another advantage of the method disclosed herein is that it enables biofilm delamination from substrates rather than merely inhibiting biofilm growth or destroying biofilm without removal as in known methods, which can be important for multiple use and long shelf life of substrates.

The method disclosed herein may be especially important for those persons who are in business trips, camping, travelling as well as for patients who have dementia or diseases that impede self-care.

Also provided herein is a coated substrate comprising a substrate, and metal particles on the surface of the substrate. In some embodiments, the metal particles have an average diameter in a range of 200 nm to 2000 nm, wherein a shortest distance between exterior surfaces of adjacent metal particles is at least 10 nm. In some embodiments, a density of the metal particles is in a range of 0.1 to 30 metal particles per 1 μm² of the substrate surface.

Particles on Dental Restorations—In one embodiment, the coating disclosed herein is applied to dental restorations. Specific roughening restorations, surface and chemical solutions of reducing agents and metal ions are used for short term (minutes) soaking of dental restorations in baths so to bind the coating to the surfaces of zirconium or silicate based dental restorations such as: crowns/veneers/inlays/onlays/bridges, etc. In some embodiments, the coating particles are silver. In some embodiments, the coating particles include other metals usually applied in dentistry, e.g. gold, copper, platinum, palladium, nickel, chromium, cobalt, titanium, stainless steel, aluminum, zinc. Coating of restorations can be performed in a dental lab or in a dental office setting before the restoration is permanently cemented into the oral environment. The resulting particles are then irradiated with the light source intraorally at home to cause pathogenic formation delamination from the dental restoration.

Particles on Natural Teeth—In one embodiment, the coating disclosed herein is applied to natural teeth. A solution of reducing agents and metal ions for short term (minutes) is used to provide growth of particles on natural tooth hydroxyapatite. Direct growth of metal particles with disclosed parameters on natural teeth from a simple ionic solution of silver is described herein. This universal coating process can be applied in the dentist office by a certified professional using standard techniques for isolation techniques of the tooth surfaces. This coating is to be applied biannually on the natural tooth surfaces to achieve biofilm delamination when it is intraorally radiated with the light source at home.

Particles in Dental Glaze—In one embodiment, a dental glaze is manufactured with particles to be applied in the dental lab or dental office setting to an unfinished zirconium oxide, titanium oxide, silicate or other dental restoration. The particles are reduced from ionic to atomic form in solution of their ions. For silver particles, silver nitrate is dissolved in an aqueous solution to dissociate to silver ions and create a suspension of the silver ions in solution. The metal particles exist in a dispersion phase together with a separate substance in the continuous phase to form a colloid solution including a colloidal formation of metal particles dispersed in a typical dental glaze or other coating used in dentistry as a sealant or barrier applied to natural or synthetic surfaces. In such an application, the solution comprising the glaze and particles is applied to restorations by traditional techniques, then finally fired and sintered. After treatment, the coated dental surface is prepared for subsequent delamination of biofilm pathogenic formations by intraoral light radiation.

Particles for Periodontal Patient Benefits—In one embodiment, particles on periodontally involved teeth can achieve pathogenic formation delamination from the indirect intraoral light source (scattered light beyond 3-5 mm from focal area). The delamination is effective for patients suffering from deeper periodontal pockets to eliminate the toxic plaque and tartar in the deeper recesses under the gumline. Therefore, tooth surfaces 3-5 mm below the crest of the gumline will also exhibit plaque/tartar delamination with particles and light source illumination. Delamination keeps plaque levels to a minimum for these compromised patients with hard to reach areas.

Particles for Dental Implant Patient Benefits—In one embodiment, particles can be either grown, using previously mentioned various bath solutions or sintered using previously mentioned glaze process for zirconium or silicate based dental implant restorations such as implant supported all on 4's/implant hybrid restorations/single unit implant crowns/implant bridges

Particles for Orthodontic Patient Benefits—In one embodiment, particles are placed on all orthodontic appliances that are going to be temporarily fixed into the oral environment. Oral hygiene is tremendously challenging for orthodontic patients due to the rapid accumulation of food particles in the wires and brackets used to engage the teeth in orthodontic therapy. Use of intra oral light therapy to more easily delaminate plaque and food cells (dead cells) from the surfaces of the appliances upon light excitation is highly beneficial to the patient. This can help keep their caries status in check and lead to better orthodontic outcomes. Metals for orthodontic therapy can be the most common types of metals that are used in dentistry and their usages are alloys of this list: gold (crown), silver (amalgam fill), copper (amalgam fil), platinum (crown), palladium (crown), nickel (amalgam fill/implant/orthowire), chromium (crown/amalgam fill), cobalt (crown/partial denture), titanium (implant/orthowire), stainless steel (ortho bracket), aluminum (temporary crown) and combinations thereof.

Particles for Endodontic Patient Benefits—In one embodiment, application of particles can also be extended into the pulp chamber and canal spaces of dental root canals. Once a narrow “glide path” has been reached to “corrected working length” on an endodontically treated tooth, the particles are introduced into the chamber. The solutions for growth of particles mixed with an EDTA solution, penetrates all the tubules of the primary and secondary canal spaces. The insertion of a tiny optical fiber with diameter below that of root canal coupled to a directed low power light source will provide delamination of the organic compounds (necrotic tissues, abscess) from the internal anatomy of the tubules and canal systems. One can then correspondingly flush out the necrotic and infected biomass.

Because scattered light provides delamination effects up to 3-5 mm beyond the focal light source, the clinician can easily reach into the deeper tubules and secondary canals. This means less natural tooth structure is removed to achieve the disinfection of the tortuous secondary canals of complex roots. Since the tooth will be minimally debrided, this technique preserves the structural integrity and increases its future life expectancy. In addition, a final high-powered burst of energy can be applied through the optical fiber coupling with another light source, e.g. powerful-pulsed infrared laser (over 5 mW), to cause intense heat effectively collapsing the tubules. This can possibly replace the current use of guttacore obturation techniques.

Particles for Universal Adult Patient Benefits—In one embodiment, the coating disclosed herein can be safely applied to all dental surfaces and restorations thereby used universally. The disclosed method can both augment and replace some current modalities of oral hygiene and reduce the need for traditional tooth brushing, flossing, water picking and dental office scalings.

The method disclosed herein can help prevent dental caries and keep patients healthy. For example, twice-daily intra oral light application for 30 seconds and then swishing with an expectorant can remove oral biofilm.

### Other Industries:

1. Nanoparticle Dishwasher—using the same ceramic coating concept and a modulated light source, one can apply the light to the surface coated plates, cups, cutlery, pots dishes, etc. and with minimal water flush, you create a much faster cycle dishwasher cycle. The particle sizes and light power density will have to be calibrated to achieve an ideal result for this industrial application.

2. Nanoparticle Autoclaves—using the same ceramic coating concept and a modulated light source, one can apply the light to the surface coated ceramic based surgical instruments and tools. This can become a rapid autoclave where all organisms and spores can be rapidly easily removed without the use of high temperatures needed to cook and destroy the cellular organisms. The particle sizes and light power density will have to be calibrated to achieve an ideal result for this industrial application.

In certain embodiments, particles can be incorporated in root canals and tubules for delamination of necrotic tissues.

In some embodiments, the method includes using:

a) silver particles with sizes from 200 nm to 2000 nm (or larger), wherein a shortest distance between exterior surfaces of adjacent silver particles is at least 10 nm, and a density of the silver particles is below 10 in 1 μm², and wherein the silver particles do not provide high frequency of free electrons oscillations leading intensive SPR and to extreme light trapping;
b) a light excitation wavelength that does not coincide with weak SPR even if it appears in the particles to avoid their heating; and
c) a low-powered light source, which does not cause significant temperature increase.

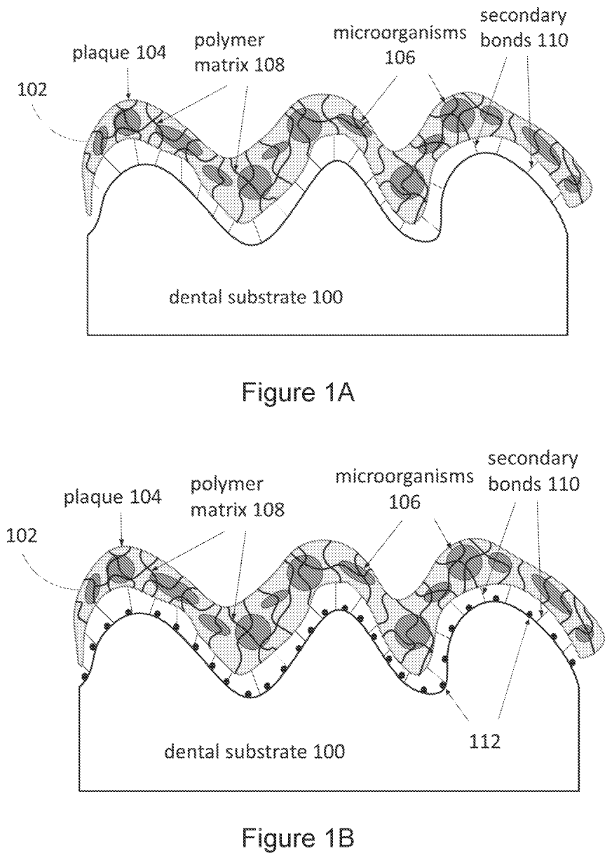

Referring to the Figures, pathogenic formations on a dental substrate can be a plaque, which is a biofilm composed of harmful species such as bacteria, viruses, fungi, dead cells of oral cavity mucosa and/or food introduced in a matrix of polysaccharides. If not removed properly, e.g., by teeth cleaning with brush twice a day, plaque can be enriched with minerals and converted in tartar. FIG. 1A depicts dental substrate 100 with pathogenic formation 102 coupled to substrate 100 with secondary bonds 110. As depicted, pathogenic formation 102 includes plaque 104 (or biofilm) with microorganisms 106 and polymer matrix 108. Plaque 104 is formed from adsorption of proteins in saliva on dental substrate 100 surface via weak secondary bonds 110 (e.g. van der Waals bonds), which are not as strong as chemical or interatomic bonds and typically appear if the distance between objects is on the Angstrom scale. Weak secondary bonds are caused by mutual attraction between the molecules of the substrate material and protein molecules. Substrate 100 can have a negative or positive charge depending on its physic-chemical properties and pH of the oral cavity environment. Proteins, which are macromolecules with differently charged endings, orient to the substrate having an oppositely charged ending. As a result, proteins from saliva form a thin viscous film on which bacteria feed and grow to create a substrate for a pathogenic formation. Microorganisms 106 (e.g., bacterial cells) immobilize on this film, capture saccharides supplied by food, and build polymer matrix 108 (e.g., polysaccharide matrix) around them. Other microorganisms 106 (viruses, dead cells, fungi, etc.) from the oral cavity environment are then easily embedded in this matrix and can use bacteria cells as hosts to live while others contain molecules for bacteria breeding. Polymer matrix 108 can also act as a barrier that protects microorganisms 106 against sterilization with any antimicrobial agents. Without wishing to be bound by theory, despite the fact that pathogenic formations are coupled to dental objects with rather weak chemical bonds, their adhesive strength is very high due to rough surface of the substrate as illustrated in FIG. 1A.

FIG. 1B depicts dental substrate 100 with metal particles 112 on the surface of substrate 100.

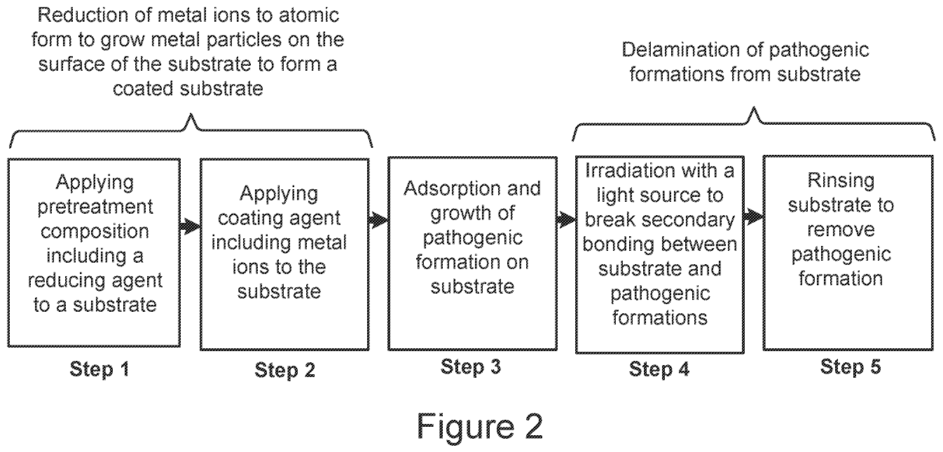

The method disclosed herein enables successive deactivation of pathogenic formations through simple removal from the dental objects, in contrast to known approaches of antibacterial agents' insertion in biofilm or between biofilm and substrate, which cause breaking chemical bonds in molecules of polysaccharides, bacteria membranes and other biofilm components. Conditions are created for the delamination and removal of pathogenic formations from a substrate using the method containing the following steps as illustrated in FIG. 2: (step 1) applying solution with reducing agent to the substrate using recipes and regimes described above, (step 2) applying solution with metal ions to the substrate using regimes described above, (step 3) waiting for self-adsorption and growth of pathogenic formations on substrate, (step 4) excitation of pathogenic formation with low power light source to break secondary bonding between substrate and pathogenic formations, and (step 5) rinsing substrate, for example by patient with water to remove (spit) pathogenic formations.

### EXAMPLES

### Coating a Substrate

The substrate is placed in a solution of metal ions, optionally with activating agent, and then reducing agent is added. When the metal ions are silver ions, there is no need for an activating agent as silver is deposited according to the silver mirror reaction. When the metal ions are not silver ions, an activating agent is added to the solution. As used herein, “activating agent” refers to ions of alternative metals having a redox potential lower than that of the depositing metal. The reduction can vary in time (2-12 h) depending on the reagents.

When the substrate is artificial teeth, short (few minutes) successive steps of their immersion into reducing agent solution and solution of silver (or other metal) ions can be used.

When the substrate is natural teeth, successive steps of brush applications to them from two different solutions containing reducing agent and silver (or other metals) ions can be used.

### Application of Coating to a Dental Crown

Chemical deposition procedure: Freshly prepared and glazed ZrO₂ crown is immersed in reducing agent solution for 1 min then rinsed with diluted ethanol solution (H₂O:C₂H₅OH=1:1 vol. ratio) or acetone for 2-5 seconds. Reducing agent solution is 0.4 M KNa tartrate water solution. After that, the crown is immersed in metal ions solution for 2 min and then rinsed with diluted ethanol solution (H₂O:C₂H₅OH=1:1 vol. ratio) or acetone for 30 seconds. For silver particles growth the following composition can be used: 0.035-0.04 M AgNO₃, 0.1 M NaOH, 0.3-0.6 M NH₃ water solution. Particles can be grown similarly under the glaze and/or on the glaze. Crowns/glazed crowns can be preliminary roughened with piranha solution (H₂SO₄: H₂O₂=3:1, keeping for 5 minutes in piranha solution at 70-80° C., then rinsing with water for 1 minute), fluoride-containing solutions (e.g., keeping for 5 minutes in solution of 5 M HCl and 1.5 M NH₄F (pH=2.8) at 21° C., then rinsing with water for 1 minute). All the processes are performed at 21° C. except treatment in piranha solution.

### Application of Coating to Natural Teeth

Natural teeth are rinsed with phosphoric acid (37%), diluted ethanol solution (H₂O:C₂H₅OH=1:1 vol. ratio) or acetone for 2-5 seconds, dried and subjected to brush applications of reducing agent solution. For silver particles growth the brush is first impregnated with 0.025 ml of the reducing 1 M glucose water solution for 10 s, then rinsed with water for 2-5 seconds and again brushed with the same reducing solution for 10 seconds and rinsed with water for 2-5 seconds. After that, the brush is impregnated with 0.025 ml of 0.035 M AgNO₃, 0.1 M NaOH, and 0.03 M NH₃ water solution and applied to the tooth for 2 minutes. Finally, teeth are rinsed with diluted ethanol solution (H₂O:C₂H₅OH=1:1 vol. ratio) or acetone for 30 seconds. All the processes can be performed at 21° C.

### Fabrication of Dental Glaze Having Silver Nanoparticles

The composition of the solution made of commercial dental glaze and particles and regimes for its application to artificial teeth and further sintering to fabricate novel glazing provide pathogenic formations delamination.

Particles are fabricated in a mixed and heated solution of silver ions with sodium citrate (100 mL of water, 150 μL of 1 M AgNO₃ water solution, and 100 μL of 1 M sodium citrate water solution). The heating should not result in solution boiling. The heating should be stopped when solution is darkened. Particle separation is performed at 6000 rpm for 10-15 minutes. Then 0.015 g of particles is mixed with 0.035 g of commercial dental glaze (any manufacturer) and 0.025 g of corresponding commercial solvent for dental glaze.

### Delamination

The pathogenic formations may be delaminated by application of light to particles as follows:
a) defocused laser (laser spot area is about 150 mm, distance between light source and crown/tooth can be up to 20 cm, time of light treatment—30 seconds), the lasers are commercially available (e.g. Oxlasers), the power is given in technical characteristics of the lasers, but it is recommended to additionally measure power density with any commercial power meter with photodetector in the point of the dental object location;
b) dental LED devices for teeth whitening (the distance between the light source and the crown/tooth is about 1-10 mm, typically power density of these devices is about 0.1-1 mW per square cm, as was measured in our experiments with pyroelectric detector head for Merlin (model 70123) in the point of the dental object location after light passing the polymer core, i.e. the source can be installed in oral cavity and used at home; examples are Dental Whitening USB charging Home Led Blue Light Tooth Whitener (model BABYTWO16), IVISMILE Teeth Whitening kit, TONGWODE (model MElY101-21).

The delamination was observed after rinsing the crown/tooth in water under vibrational and rocking modes.

### Application of Coating to Natural Teeth

The substrate was molars (wisdom teeth) of a volunteer (31-year-old woman). The sets of control dental objects not subjected to steps 1 and 2 but subjected to steps 3 (plaque growth), 4 (excitation) and 5 (rinsing); dental objects after steps 1-3, 5 but not subjected to step 4 (excitation), and dental objects subjected to all steps were prepared (step numbers according to FIG. 2).

### Application of Coating to Dental Crowns

For the crowns and molars, the process described above was used to grow silver particles. Plaque was grown on the surface of dental objects in a liquid nutrient media (agar, broth supplied by Sigma Aldrich) mixed with typical oral cavity bacteria (Streptococcus mutans, Staphylococcus aureus, Staphylococcus epidermidis, Escherichia coli from Kwik Sticks supplied by Microbiologics) under constant stirring at 36.6° C. for 72 hours. LED with 445 nm wavelength (Home Led Blue Light Tooth Whitener (model BABYTWO16)), which provided power density 0.7 mW per 4 square mm of dental object measured with power meter equipped with photodetector, was used for 30 second excitation of dental objects. Other light sources were switches off during LED treatment.

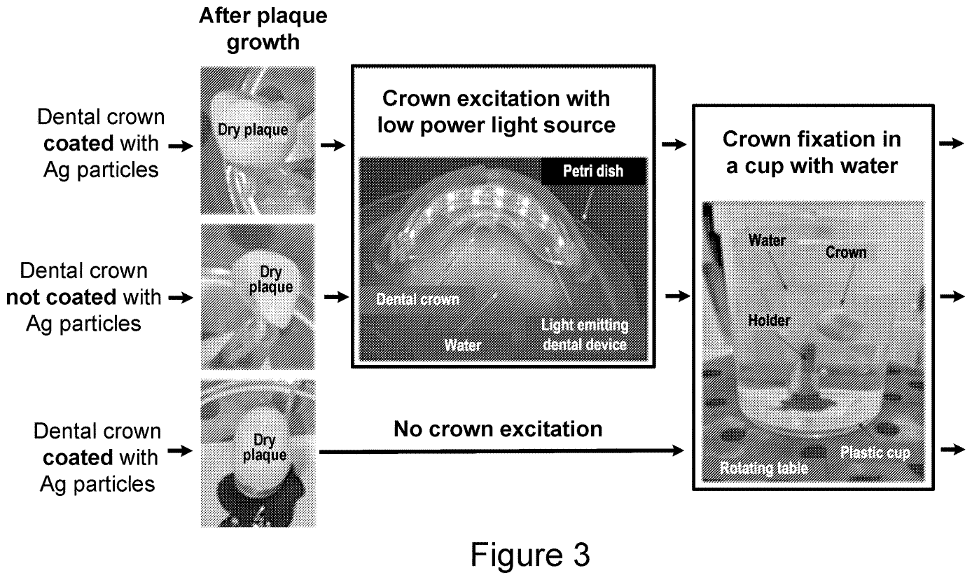

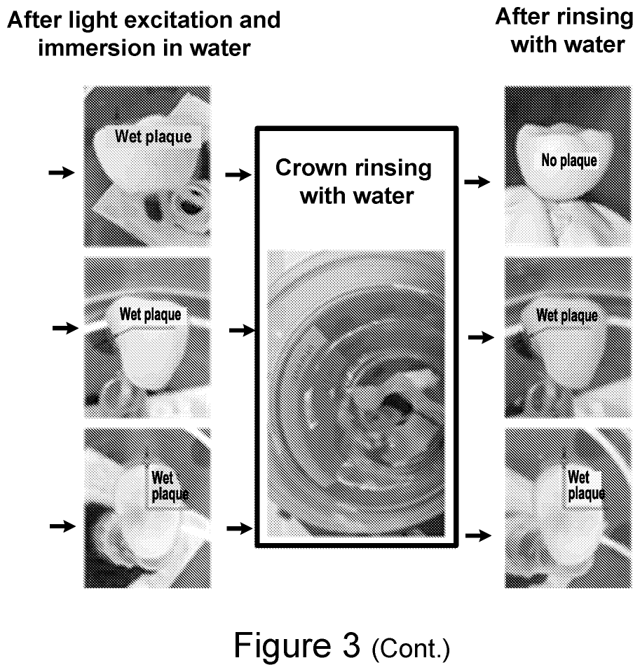

FIG. 3 shows an exemplary representation of all steps for sets of crowns. In more detail, this is a comparison of crowns subjected to all the steps of the method shown on FIG. 2 (top crown photo in each column) and those for which steps 1-2 (middle crown photo in each column) or step 4 (bottom crown photo in each column) were missed. The delamination of the plaque from the crown subjected to the proposed method is well observed (top photo, after rinsing with water) in contrast to crowns that did not contain Ag particles (middle photo, after rinsing with water) or were not excited with light (bottom photo, after rinsing with water). The same results were observed for the molars.

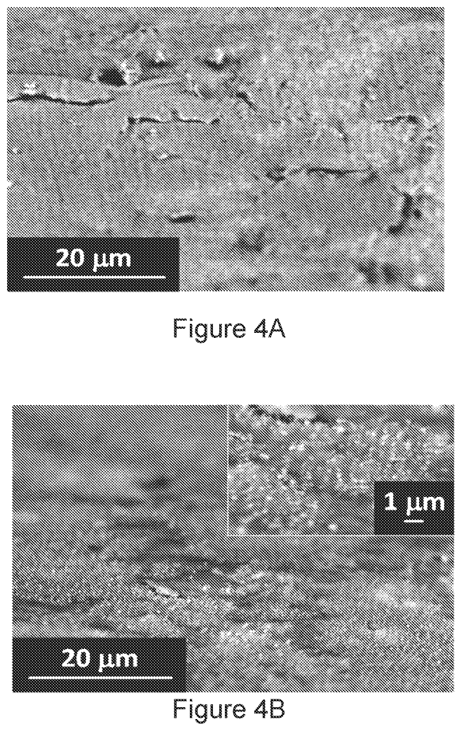

The dental surfaces before and after Ag particle growth were studied by scanning electron microscopy (SEM, Hitachi 4800). FIGS. 4A and 4B illustrate SEM top images of a crown before (FIG. 4A) and after (FIG. 4B) growth of Ag particles by chemical deposition of Ag from solution of AgNO₃, KNa tartrate, NaOH, NH₄OH, and H₂O. The diameter of Ag particles varies from 230 to 450 nm. In rare spots, they coalesced into bigger agglomerates. The number of Ag particles per 1 μm² varies from 4 to 8.

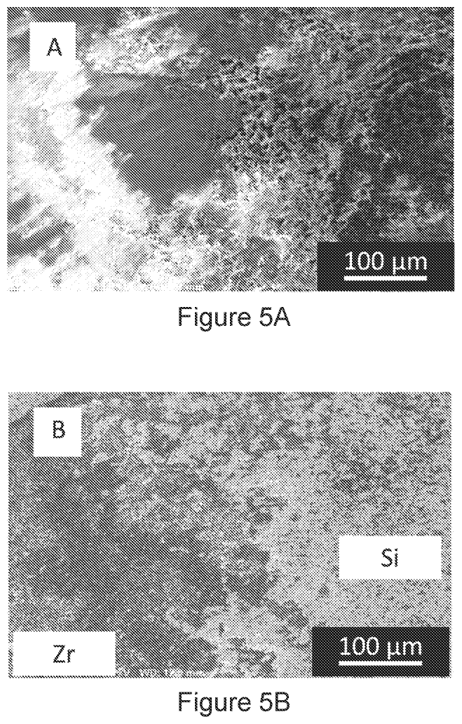

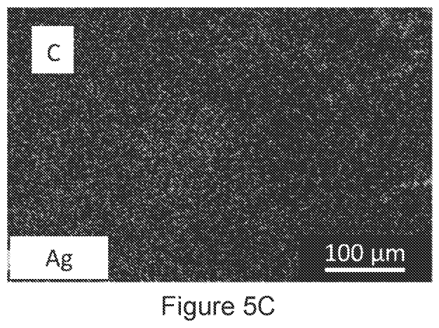

FIGS. 5A-C were made using scanning mode of energy dispersive spectrometer (EDS, Bruker) embedded into SEM for the crown, which was glazed non-completely to check if the Ag particles are grown uniformly on the glazed and non-glazed surfaces. The SEM top image is shown in FIG. 5A and the corresponding results of elemental analysis of the dental object (ZrO₂ crown) after growth of Ag particles made by EDX scanning are shown in FIGS. 5B and 5C. The crown was partially covered with SiO₂ dental glaze (light area in FIG. 5B). Presence of Zr atoms is caused by ZrO₂ composition of crown. Presence of Si atoms is caused by SiO₂ glaze on crown. Uniform distribution of Ag particles is well observed in FIG. 5C.

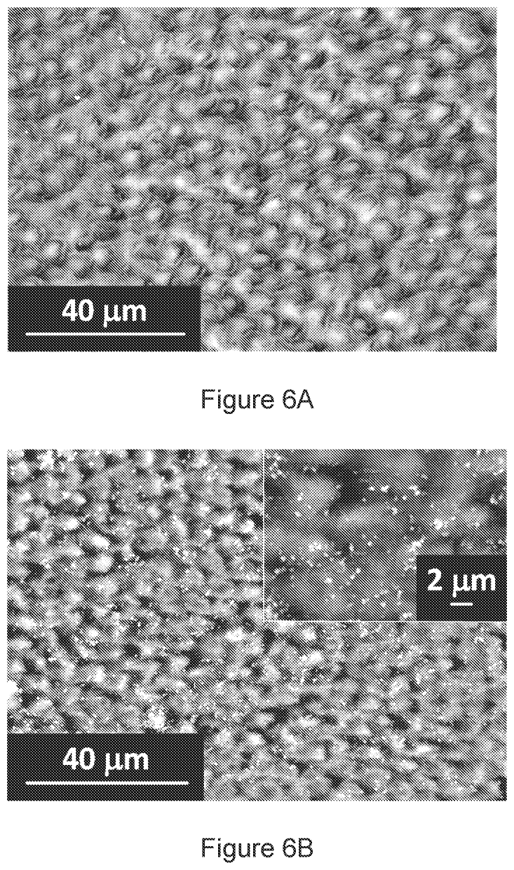

FIG. 6 illustrates SEM top images of molar before (FIG. 6A) and after (FIG. 6B) growth of Ag particles by chemical deposition of Ag from solution of AgNO₃, glucose, NaOH, NH₃, and H₂O. The size of Ag particles varies from 200 to 550 nm. Sizes of some particles are depicted in the inset to the right image and are 516 and 556 nm. In rare spots, it reaches 1-2 μm because of particles coalescence. The smoothened surface of the molar after Ag particles growth is due to pretreatment in 37% phosphoric acid typically used in dentistry. Number of Ag particles per 1 μm² varies from 1 to 10.

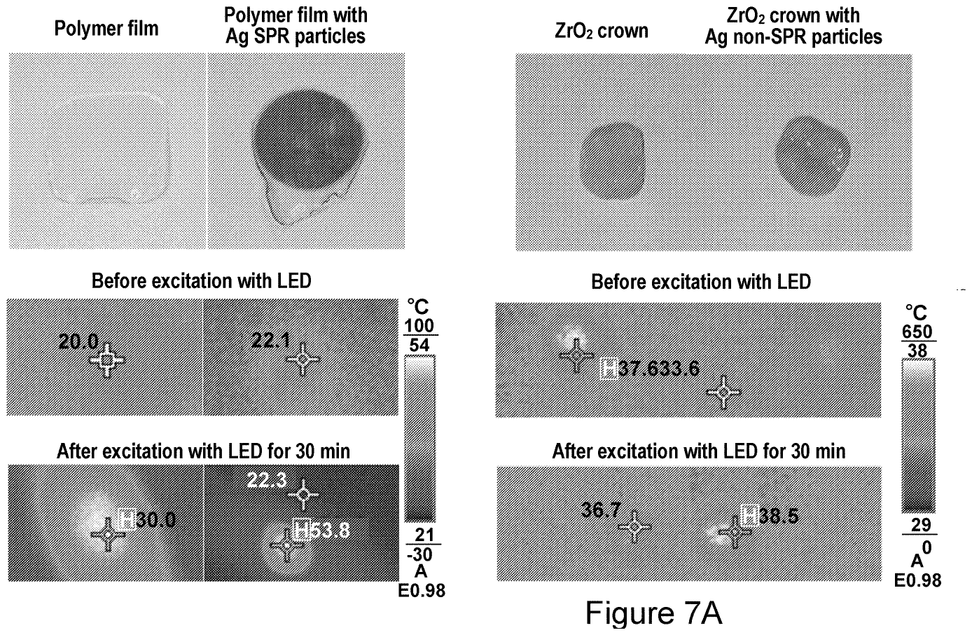

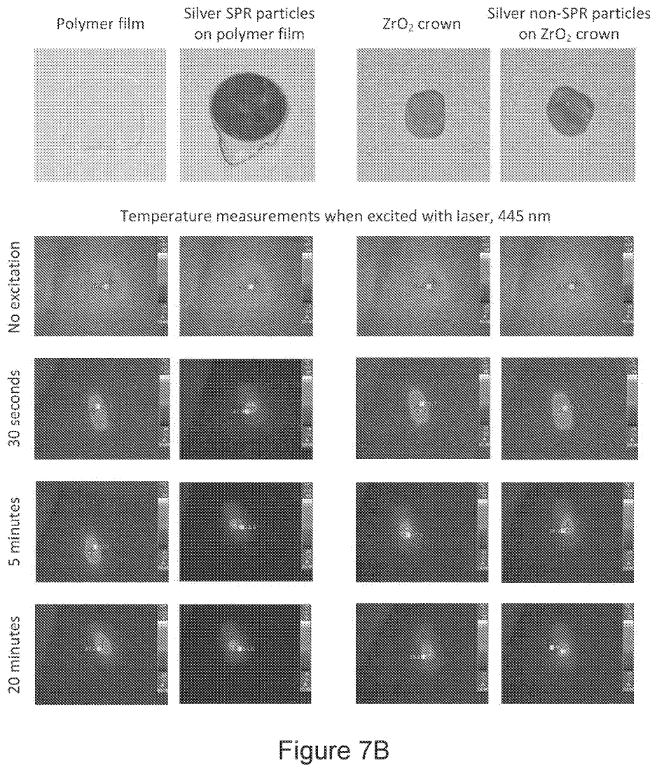

For assessment of the temperature of the particles on the surface of the dental substrate, measurements with infrared camera Testo 871 were carried out. The camera thermal sensitivity is 0.009° C. The substrate was ZrO₂ doped with yttrium oxide and the particles were silver particles presented in FIG. 4B. The silver SPR particles were prepared using regimes providing fabrication of SERS-active substrate with prominent SPR reported elsewhere. [Bandarenka, H. V., Girel, K. V., Bondarenko, V. P., Khodasevich, I. A., Panarin, A. Y., & Terekhov, S. N. (2016). Formation Regularities of Plasmonic Silver Nanostructures on Porous Silicon for Effective Surface-Enhanced Raman Scattering. Nanoscale Research Letters, 11(1). doi:10.1186/s 11671-016-1473-y]FIGS. 7A and 7B show temperature distribution images taken with the with infrared thermal imaging camera Testo 871 for silver SPR particles and silver particles on ZrO₂ doped with yttrium oxide (dental crown) upon excitation with visible LED for 30 minutes (FIG. 7A) and defocused laser for 20 minutes (FIG. 7B).

Measuring the temperature in silver particles on ZrO₂ crown upon excitation with visible LED for 30 minutes with infrared thermal imaging camera Testo 871 shows just negligible warming up by 2° C. in contrast to silver SPR particles heated up by 31° C. (FIG. 7A). Therefore, the temperature is not above trace heat resulting from interaction between the light source and an ordinary surface and is below the threshold heat needed to destroy polysaccharide bonds but is sufficient to delaminate the pathogenic formations in any area where the coated dental surface has been formed.

Measuring the temperature in silver non-SPR particles on ZrO₂ crown upon excitation with defocused laser for 20 minutes with infrared thermal imaging camera Testo 871 shows warming up by 19.9° C. from the initial temperature of 19.3° C. (FIG. 7B). This is just 0.3° C. higher that the temperature of heating the ZrO₂ crown free of silver non-SPR particles (FIG. 7B), i.e. silver non-SPR particles do not contribute to heating of the crown. The final temperature of the crown with silver non-SPR particles is 39.3° C., which is still not dangerous for the living tissues in human body. Therefore, the temperature is not above trace heat resulting from interaction between the light source and an ordinary surface and is below the threshold heat needed to destroy polysaccharide bonds but is sufficient to delaminate the pathogenic formations in any area where the coated dental surface has been formed. However, silver SPR particles on polymer heated up by 34.7° C. from the initial temperature. The final temperature of the polymer without silver SPR particles was only 37.2° C., whereas the final temperature of the polymer with SPR particles is 54° C., which is rather dangerous for living tissues.

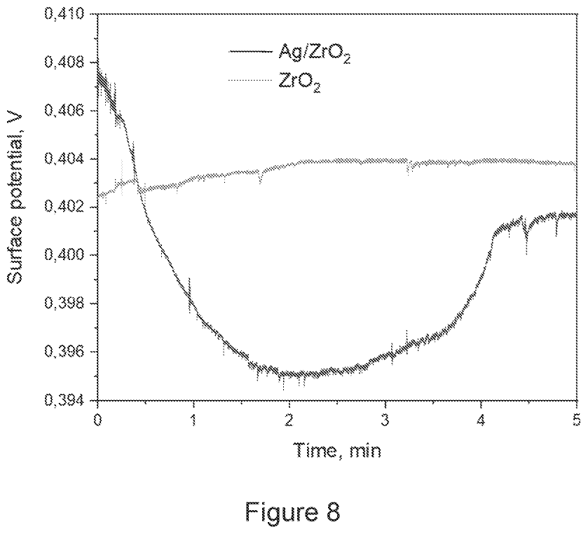

The surface potential of a zirconia dental object (crown) was studied. FIG. 8 shows the surface potential of a ZrO₂ crown and a ZrO₂ crown coated with silver particles versus time. Zero and four minute points on the time axis correspond to turning on and turning off a light emitting diode of a dental device, respectively. The silver particles on the ZrO₂ crown promote decreasing the surface potential, which is caused by contribution of charge change, in contrast to a ZrO₂ crown without silver particles having a nearly constant surface potential.

The silver particles on zirconia dental object (crown) were studied with Raman spectroscopy. This was performed via measuring SERS spectra of testing analyte (Ellman's reagent) and calculation of Raman signal enhancement factor (EF) which is below three orders of magnitude for chemical mechanism, and over three orders of magnitude for electromagnetic mechanism (thanks to SPR). SPR particles such as those described with respect to FIG. 7 were used. Ellman's reagent was selected due to its well-studied Raman spectrum and low luminescence. The blue laser (473 nm) was used to excite chemical bonds vibration in Ellman's reagent molecules and to facilitate SERS effect.

$$EF = ( {I}_{SERS} \cdot {C}_{RS} ) / ( {I}_{RS} \cdot {C}_{SERS} ) ,$$

where I_{SERS}—intensity of particular band of analyte in SERS spectrum, C_{SERS}—minimal concentration of analyte at which it is detected with SERS-active substrate (silver particles), I_{RS}—intensity of particular band of analyte in Raman spectrum, C_{RS}—minimal concentration of analyte at which it is detected without SERS-active substrate.

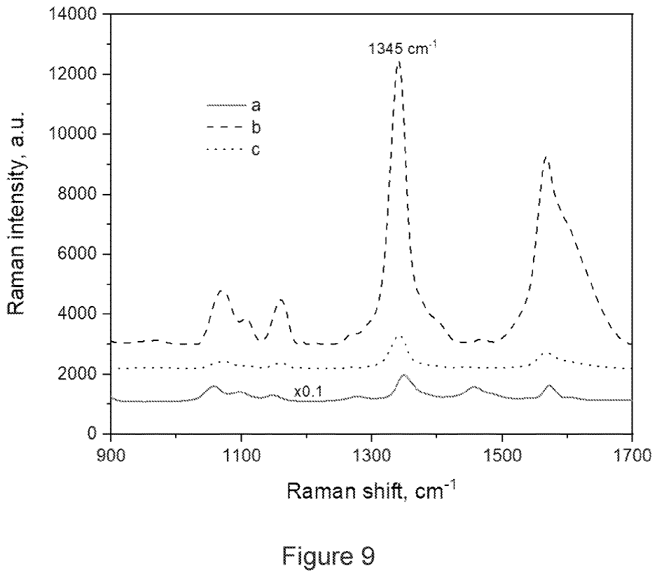

EF was calculated using C_{RS}=10⁻¹ M and C_{SERS}=10⁻⁵ M. FIG. 9 shows Raman (a) and SERS spectra (b and c) of Ellman's reagent (5,5′-dithio-bis-[2-nitrobenzoic acid]) at concentration (a) 10⁻¹ M on glass, (b) 10⁻⁵ M on Ag SPR particles and (c) Ag non-SPR particles on ZrO₂ dental object (crown). Intensity at 1345 cm⁻¹ band for Raman spectrum of Ellman's reagent is 9280 a.u., for SERS spectra registered with SPR particles—9510 a.u., non-SPR particles—1290 a.u. Calculations resulted in EF=1.025-105 for SPR particles and 1.139-103 for non-SPR particles. The EF of particles is typical for SERS induced by chemical mechanism supported by effects (e.g. CT).

### Fabrication of Plasmonic Silver Nanostructures

While this fabrication can be employed to a large set of materials, an approach for silicon- and zirconia-based substrates is described.

Silicon-based substrates. Highly doped monocrystalline n-Si wafers were used as initial substrates. Two approaches were used to fabricate plasmonic coatings.

1. In liquid solutions: Fabrication of plasmonic silver nanostructures was performed via immersion deposition of silver on porous silicon described elsewhere [1].

These nanostructures demonstrate prominent plasmonic property and are applied for chemical and biosensing by surface enhanced Raman scattering (SERS) spectroscopy.

2. In low-vacuum environment: Si wafer was diced in 10×10 cm samples that were cleaned in acetone, isopropanol and deionized water. Then the samples' surface was coated with a silver film of 20 nm thickness by magnetron sputtering and annealed in an argon atmosphere at 600° C. for 5 min to provide silver film dewetting in nanoparticles.

Zirconia-based substrates. Yttrium doped zirconia dental crowns were used as initial substrates. Each crown was cleaned in acetone, isopropanol and deionized water.

1. In liquid solutions, immersion technique: The crown was immersed in a liquid solution composed of deionized water (100 ml), silver nitrate (0.22 g), ethyl alcohol (6 ml) and hydrofluoric acid (7.26 ml). Single crown coating requires 5 ml of solution for immersion deposition of silver. The immersion lasts for 2 h. After deposition the crown is removed from the solution, triple rinsed with deionized water and dried with airflow.

2. In liquid solutions, chemical technique: This recipe was developed to fabricate plasmonic coating avoiding hydrofluoric acid use due to its safety concerns. The crown is immersed in a liquid solution composed of ammonia solution (50 ml), deionized water (50 ml), silver nitrate (0.02 M), sodium hydroxide (0.2 M) and potassium sodium tartrate (0.04 M).

3. In low-vacuum environment: The crown was subjected to magnetic sputtering of silver film with thickness of 50 nm under constant rotation. After silver sputtering the crown was immersed in diluted nitric acid (10%) for 0.5 h, and then removed, triple rinsed with deionized water, and dried with airflow. After that, the crown was annealed at 700° C. for 5 min.

### Evaluation of Plasmonic Strength of the Samples

SERS-activity of the silver-coated substrates was evaluated to reveal their plasmonic strength. It is generally accepted that the ability to provide over three orders of magnitude enhancement of Raman signal from molecules adsorbed on metallic nanoparticles is caused by their prominent plasmonic property. Each silver-coated substrate (silicon- and zirconia based) was kept in a 10⁻⁶ M DTNB (Ellman's reagent) or R6G (organic dye) aqueous solution for 2 h, then rinsed with deionized water and air-dried. SERS-spectra were recorded using confocal Raman microscope Witec equipped with a green laser (532 nm). The laser was focused on the substrate surface via ×100 objective. An excitation time of 1 s was the same for all the samples. If the spectra measured revealed a clear signature of the molecules, then it was called a “plasmonic active substrate”.

### Fabrication of Model Biofilm (Polysaccharide Film)

The model biofilm is produced ex-situ in a Petri dish to mimic the real scenario inside a bio-organism. The silicon- or zirconia-based substrate coated with plasmonic nanoparticles was placed in sterilized Petri dish, which was then filled with a liquid agar (e.g. Trypsin soy agar) at 45-75° C. to be over the substrate for 0.1-0.5 mm. After that, the Petri dish was kept for 10 min at 21° C. for agar gelation.

The silicon- or zirconia-based substrate coated with plasmonic nanoparticles was placed in sterilized Petri dish, which is then filled with liquid agar (e.g. blood agar) mixed with target bacteria (e.g. s. mutans) at 45-55° C. to be over the substrate for 0.1-0.5 mm. The mixture was prepared by dropping 0.1 ml of the bacteria-containing water solution (2·10⁴ CFU/ml) into 100 ml of liquid agar and subsequent magnetic stirring for 1 min. After that, the Petri dish was kept for 10 min at 21° C. for agar gelation and then for 72 h at 30° C. for bacteria growth in the model biofilm.

### Example A: Delamination of the Biofilm

Activation of the plasmonic coating: The silicon- or zirconia-based substrate coated with plasmonic nanoparticles and model biofilm was subjected to continuous excitation from the light source (e.g. laser) with wavelength 445-450 nm. The spot size irradiated was 5×10 mm² and the laser power was 1.6 W. The light spot completely covered the substrate surface for 30 sec. The distance between the substrate surface and the light source was 18 cm.

Washing the substrates: After excitation with light, the silicon- or zirconia-based substrates coated with plasmonic nanoparticles and model biofilm were cut out from the agar Petri dish and fixed with double-sided tape in sterile Petri dish, which then was filled with deionized water to completely cover the substrates' surface. The Petri dish was placed on platform that rocked with 100-120 RPM speed. In the rocking station, the water moves left to right in the Petri dish similarly to how a washing machine removes soil from clothes, exerting friction against the biofilm which is then forced to delaminate. If model biofilm delaminated from the substrate, the time spent for the delamination was registered. The test is standardized by selecting the Petri dish size, the water fill level and the rocking frequency and amplitude. Delamination was observed for light excited substrates coated with plasmonic silver nanoparticles.

Testing for Bacteria Presence after Delamination

After washing the model biofilms not delaminated from the substrates were removed with scalpel. All the substrates were rinsed deionized water for 30 s and then swabbed. The swab was applied to the surface of agar (e.g. blood agar) Petri dish, which then was kept for 24 h at 30° C. for bacteria growth.

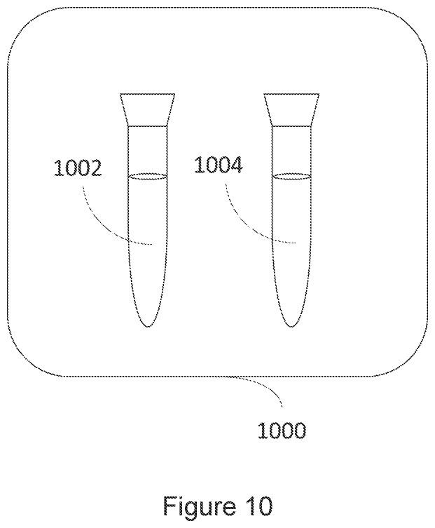

Also provided herein is a kit including a pretreatment composition comprising a reducing agent, and a coating agent comprising metal ions, all as described herein. In some embodiments, the coating agent comprises silver ions. In some embodiments, the coating agent comprises silver nitrate. In some embodiments, the reducing agent comprises potassium sodium tartrate. FIG. 10 depicts an example of a kit 1000 with pretreatment composition 1002 and coating agent 1004.

In some embodiments, the kit further comprises a light source configured to be inserted in the mouth of a user. In some embodiments, the kit further comprises an activating agent. In some embodiments, the kit further comprises a measuring container or measuring equipment that can be used to measure the solutions. In some embodiments, the kit further comprises an immersion container suitable for immersion of the substrate, such as a dental substrate. In some embodiments, the kit further comprises a brush for applying the coating. For example, a brush suitable to apply the coating to natural teeth.

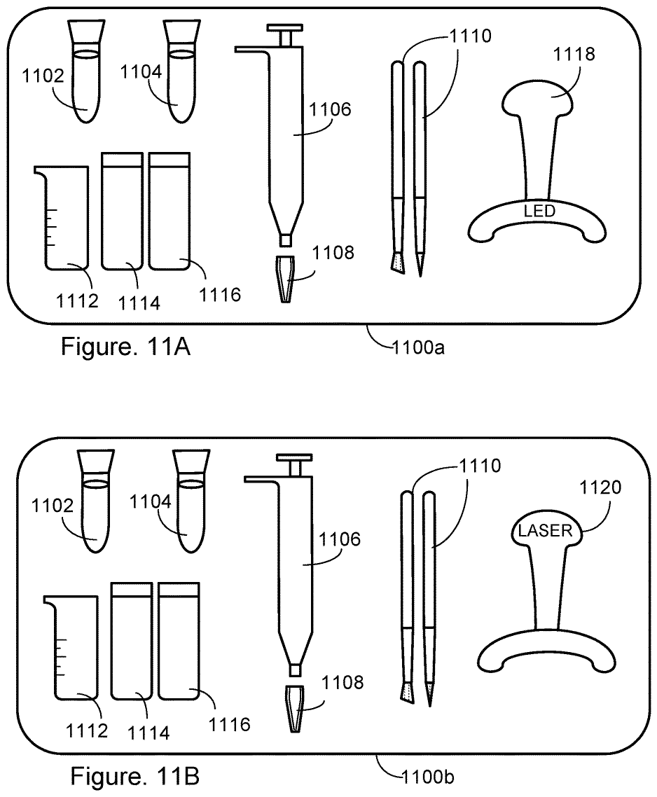

FIGS. 11A and 11B depict examples of a kit 1100a and 1100b, respectively, with a pretreatment composition 1102, a coating agent 1104, and other optional components. A pipette 1106 is optionally included for dosing the solutions. One or more nozzles 1108 for the pipette are optionally included. One or more brushes 1110 are optionally included, for example for applying a solution to a dental substrate in the mouth. Measuring containers for rinsing brushes and dental objects after coating formation are optionally included, such as measuring container 1112, a container with distilled water 1114, and a container with ethanol 1116. A light source 1118 or 1120 configured to be inserted in the mouth of a user is optionally included. FIG. 1100A depicts an example of a kit 1100a having an LED light source 1118. In some embodiments, the LED light source 1118 is included for use by a patient, for example, the light source 1118 is supplied by a dental lab after the formation of the coating for the patient to use in their home. FIG. 1100B depicts an example of a kit 1100b having a laser light source 1120. In some embodiments, the laser light source 1120 is used by a professional, such as a dentist for cleaning at the dental lab.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A method of cleaning a substrate, the method comprising:
irradiating a pathogenic formation on a surface of the substrate with radiation in a wavelength range of 350 nm to 800 nm, wherein the substrate comprises metal particles on the surface of the substrate, and wherein a highest temperature of the metal particles during the irradiation is less than or equal to 45° C.; and
delaminating the pathogenic formation from the substrate.

2. The method of claim 1, wherein an average diameter of the metal particles is in a range between 200 nm and 2000 nm.

3. The method of claim 1, wherein the metal particles comprise silver.

4. The method of claim 1, wherein a shortest distance between exterior surfaces of adjacent metal particles is at least 10 nm.

5. The method of claim 1, wherein a density of the metal particles is in a range of 0.1 to 30 metal particles per 1 μm² of the substrate surface.

6. The method of claim 1, wherein a density of the metal particles is in a range of 1 to 10 metal particles per 1 μm² of the substrate surface.

7. (canceled)

8. (canceled)

9. The method of claim 1, wherein a density of the metal particles is in a range of 1 to 10 metal particles per 1 μm² of the substrate surface when the metal particles have an average diameter in a range of 200 nm to 1000 nm.

10. The method of claim 9, wherein a density of the metal particles is in a range of 1 to 10 metal particles per 1 μm² of the substrate surface when the metal particles have an average diameter in a range of 200 nm to 600 nm.

11. The method of claim 10, wherein a density of the metal particles is in a range of 3 to 5 particles per 1 μm² of the substrate surface when the metal particles have an average diameter in a range of 400 nm to 500 nm.

12. The method of claim 10, wherein a density of the metal particles is in a range of 5 to 10 particles per 1 μm² of the substrate surface when the metal particles have an average diameter in a range of 200 nm to 300 nm.

13. The method of claim 1, wherein the irradiating occurs for a length of time in in a range of about 0.1 seconds to about 30 minutes.

14. The method of claim 1, wherein the irradiating increases a temperature of the metal particles by 25° C. or less, 20° C. or less, 15° C. or less, 10° C. or 5° C. or less.

15. The method of claim 1, wherein, during the irradiating, the metal particles do not undergo surface plasmon resonance.

16. The method of claim 1, further comprising disposing the metal particles on the substrate.

17. The method of claim 16, wherein disposing the metal particles on the substrate comprises spraying the metal particles on the substrate, immersion deposition, or chemical deposition.

18. (canceled)

19. The method of claim 1, wherein the substrate is a natural tooth, a dental implant, or a dental crown.

20. (canceled)

21. The method of claim 1, wherein delaminating comprises rinsing the substrate to remove the pathogenic formation from the substrate.

22. The method of any claim 1, wherein the pathogenic formation comprises a biofilm.

23. A coated substrate comprising:
a substrate; and
metal particles on the surface of the substrate, the metal particles having an average diameter in a range of 200 nm to 2000 nm, wherein a shortest distance between exterior surfaces of adjacent metal nanoparticles is at least 10 nm, and a density of the metal particles is in a range of 0.1 to 30 metal particles per 1 μm² of the substrate surface.

24. A kit comprising:
a pretreatment composition comprising a reducing agent; and
a coating agent comprising metal ions.

25-27. (canceled)
